(19) United States
(12) Patent Application Publication
(10) Pub. No.: US 2018/0193659 A1
(43) Pub. Date: Jul. 12, 2018

(54) PHOTODYNAMIC THERAPY LIGHT IRRADIATING DEVICE AND LIGHT IRRADIATING METHOD

(71) Applicants: Public University Corporation Nagoya City University, Nagoya-city, Aichi (JP); Ushio Denki Kabushiki Kaisha, Tokyo (JP)

(72) Inventors: Akimichi MORITA, Aichi (JP); Hideyuki MASUDA, Tokyo (JP); Makoto KIMURA, Tokyo (JP)

(73) Assignees: Public University Corporation Nagoya City University, Nagoya-city, Aichi (JP); Ushio Denki Kabushiki Kaisha, Tokyo (JP)

(21) Appl. No.: 15/737,398

(22) PCT Filed: Mar. 25, 2016
(86) PCT No.: PCT/JP2016/059566

§ 371 Date: Dec. 18, 2017

(30) Foreign Application Priority Data
Jun. 24, 2015 (JP) 2015-126336

(51) Int. Cl. A61N 5/06; A61K 41/00

(52) U.S. Cl. CPC A61N 5/062; A61N 2005/0652; A61K 41/0057; A61N 5/0616

(57) ABSTRACT

Provided are a photodynamic therapy light irradiating device and a light irradiating method that can achieve an excellent therapeutic effect by short-time light irradiation. The photodynamic therapy light irradiating device of the present invention includes a light source part having a first LED element having a peak wavelength within a wavelength range of 400 to 420 nm and a second LED element having a peak wavelength within a wavelength range of 500 to 520 nm, and a control unit for controlling outputs of the first LED element and the second LED element. When the first LED element and the second LED element that constitute the light source part are turned on, one irradiated site is irradiated with light from the first LED element and light from the second LED element.

### TECHNICAL FIELD

The present invention relates to a photodynamic therapy light irradiating device and a light irradiating method.

### BACKGROUND ART

Conventionally, a photodynamic therapy (hereinafter also referred to as “PDT”) has been known as one of therapies using light. The PDT is a therapy using properties of a photosensitizer having affinity with a lesion in the living body (lesion abnormal tissue), for example, specific accumulation of the photosensitizer in the lesion. The PDT will be specifically described. In the PDT, the photosensitizer or a precursor thereof is administered to the living body, and then the photosensitizer (including a photosensitizer synthesized from the precursor of the photosensitizer in the living body) is irradiated with light (visible light) to produce reactive oxygen species in tissues. Only the lesion abnormal tissue is selectively destroyed using the reactive oxygen species. In recent years, the PDT is widely used in the field of dermatology for treatments of neoplastic lesions such as actinic keratosis, Bowen disease, Paget disease, and basal cell carcinoma, severe acne vulgaris, sebaceous hyperplasia, and intractable verruca.

In a photodynamic therapy light irradiating device for performing such a PDT (hereinafter also referred to as “PDT device”), a laser light source having a wavelength of 600 to 700 nm is generally used as a light source. The laser light source has a high luminance and a small irradiation area (spot diameter). Therefore, the laser light source has merits such as easy design of a device using a transmission optical element such as a fiber. Accordingly, the laser light source is efficient for a disease in which the range of lesion is small. However, the range of lesion in diseases in dermatology, typified by actinic keratosis, Bowen disease, basal cell carcinoma, and acne, is large in many cases. Therefore, when the PDT device using the laser light source having a small irradiation area is used, there is a problem in which the irradiation time for treatment is long.

A PDT device using a lamp typified by a xenon lamp or a metal halide lamp as alight source is developed and marketed. In the PDT device using a lamp as a light source, infrared light is radiated from the lamp. Therefore, there are problems caused by the infrared light, for example, a problem in which a heat sensation occurs in an irradiated region.

In recent years, in order to solve these problems, a PDT device using an LED element as a light source has been proposed (for example, see Patent Literature 1).

Specifically, Patent Literature 1 discloses a PDT device having two kinds of LED elements emitting light having different wavelength ranges as a light source. In the PDT device, the same irradiated site is irradiated simultaneously with two different kinds of light in a pulse shape from the two kinds of LED elements. In this PDT device, the two different kinds of light simultaneously emitted are light having a wavelength range coinciding with the maximum absorption peak wavelength to which a used photosensitizer is sensitive (specifically, a wavelength within a range of 400 to 550 nm, hereinafter also referred to as “sensitive wavelength range”) and light having a wavelength range other than the sensitive wavelength range (specifically, a wavelength within a range of 590 to 690 nm). In this PDT device, it is necessary that as one of the two kinds of LED elements, an LED element emitting light having a wavelength range coinciding with a sensitive wavelength range of a photosensitizer accumulated in a lesion be selectively used, and as the other, an LED element emitting light having a wavelength range other than the sensitive wavelength range be selectively used.

### CITATION LIST

### Patent Literature

Patent Literature 1: Japanese Patent Application Laid-Open No. 2008-237618

### SUMMARY OF INVENTION

### Technical Problem

Of a photosensitizer and a precursor thereof, δ-aminolevulinic acid (5-ALA) which was finally approved as a medical product (pharmaceutical approval) in July, 2014 in Japan, or the like has been newly used. Herein, the “δ-aminolevulinic acid” is the precursor of the photosensitizer, and does not have photosensitivity in itself. Protoporphyrin IX (PpIX) synthesized from the δ-aminolevulinic acid through an enzymatic reaction functions as the photosensitizer. Therefore, a useful wavelength range of irradiation light for effective treatment using a novel photosensitizer is not known in an actual clinical setting.

Herein, protoporphyrin IX (PpIX) has an absorption spectrum represented by a dashed line in FIG. 14, and has adsorption peaks at wavelengths of 410 nm, 510 nm, 545 nm, 580 nm, and 630 nm. The absorbance is increased in the order of light having a wavelength of 410 nm, light having a wavelength of 510 nm, light having a wavelength of 545 nm, light having a wavelength of 580 nm, and light having a wavelength of 630 nm. The body penetration of these beams of light is increased in the order of the light having a wavelength of 410 nm, the light having a wavelength of 510 nm, the light having a wavelength of 545 nm, the light having a wavelength of 580 nm, and the light having a wavelength of 630 nm.

FIG. 14 shows the absorption spectrum of protoporphyrin IX and spectra showing the light intensities of five kinds of LED elements each having a peak wavelength corresponding to each of the adsorption peaks of the protoporphyrin IX. The spectra are represented by curves (a) to (e). The curve (a) is the spectrum showing the light intensity of an LED element having a peak wavelength at 405 nm. The curve (b) is the spectrum showing the light intensity of an LED element having a peak wavelength at 505 nm. The curve (c) is the spectrum showing the light intensity of an LED element having a peak wavelength at 545 nm. The curve (d) is the spectrum showing the light intensity of an LED element having a peak wavelength at 570 nm. The curve (e) is the spectrum showing the light intensity of an LED element having a peak wavelength at 635 nm.

In view of the foregoing circumstances, the inventors of the present invention have intensively studied a PDT device using an LED element as a light source. As a result, the inventors have found that even when protoporphyrin IX is used as a photosensitizer, an excellent healing effect by a PDT is obtained by using two kinds of LED elements each having a peak wavelength within a specific wavelength range in combination.

As described above, the present invention has been made as the results of intensive studies by the inventors. The present invention has as its object the provision of a photodynamic therapy light irradiating device and a light irradiating method that can achieve an excellent therapeutic effect by short-time light irradiation.

### Solution to Problem

A photodynamic therapy light irradiating device of the present invention includes:

a light source part having a first LED element having a peak wavelength within a wavelength range of 400 to 420 nm and a second LED element having a peak wavelength within a wavelength range of 500 to 520 nm, and

a control unit that controls outputs of the first LED element and the second LED element, wherein

by operating both of the first LED element and the second LED element that constitute the light source part together, one irradiated site is irradiated with light from the first LED element and light from the second LED element.

In the photodynamic therapy light irradiating device of the present invention, it is preferable that the energy of the light from the second LED element is not lower than the energy of the light from the first LED element.

In the photodynamic therapy light irradiating device of the present invention, it is preferable that the control unit has an irradiation energy adjustment mechanism that controls the light from the first LED element and the light from the second LED element using pulse width modulation and the off time in the pulse width modulation is not more than 4 μs.

A first light irradiating method of the present invention is a light irradiating method for a photodynamic therapy including irradiating one irradiated site with light having a peak wavelength within a wavelength range of 400 to 420 nm from a first LED element and light having a peak wavelength within a wavelength range of 500 to 520 nm from a second LED element, wherein

an output of the light having the peak wavelength within the wavelength range of 400 to 420 nm from the first LED element is higher than an output of the light having the peak wavelength within the wavelength range of 500 to 520 nm from the second LED element.

A second light irradiating method of the present invention is a light irradiating method for a photodynamic therapy including irradiating one irradiated site with light having a peak wavelength within a wavelength range of 400 to 420 nm from a first LED element and light having a peak wavelength within a wavelength range of 500 to 520 nm from a second LED element, wherein

an output of the light having the peak wavelength within the wavelength range of 500 to 520 nm from the second LED element is higher than an output of the light having the peak wavelength within the wavelength range of 400 to 420 nm from the first LED element.

### Advantageous Effects of Invention

In the photodynamic therapy light irradiating device of the present invention, a light source part has a first LED element having a peak wavelength within a wavelength range of 400 to 420 nm and a second LED element having a peak wavelength within a wavelength range of 500 to 520 nm. When one irradiated site is irradiated simultaneously with light from the first LED element and light from the second LED element, the amount of irradiation (integrated light amount) necessary for treatment can be decreased as compared with a case where the irradiated site is irradiated each separately with the light from the first LED element and the light from the second LED element. Therefore, the irradiation time necessary for the treatment can be shortened.

Consequently, according to the photodynamic therapy light irradiating device of the present invention, an excellent therapeutic effect can be achieved by short-time light irradiation.

The first and second light irradiating methods of the present invention are a method for performing a photodynamic therapy by irradiation of one irradiated site with the light having a peak wavelength within a wavelength range of 400 to 420 nm from the first LED element and the light having a peak wavelength within a wavelength range of 500 to 520 nm from the second LED element. In the first light irradiating method of the present invention, the output of the light having the peak wavelength within the wavelength range of 400 to 420 nm from the first LED element is made higher than that of the light having the peak wavelength within the wavelength range of 500 to 520 nm from the second LED element. In the second light irradiating method of the present invention, the output of the light having the peak wavelength within the wavelength range of 500 to 520 nm from the second LED element is made higher than that of the light having the peak wavelength within the wavelength range of 400 to 420 nm from the first LED element.

According to the first light irradiating method of the present invention, a high therapeutic effect can be obtained by short-time light irradiation for a disease in which a lesion is present at a comparatively shallow position in the living body.

According to the second light irradiating method of the present invention, a high therapeutic effect can be obtained by short-time light irradiation for a disease in which a lesion is present at a comparatively deep position in the living body.

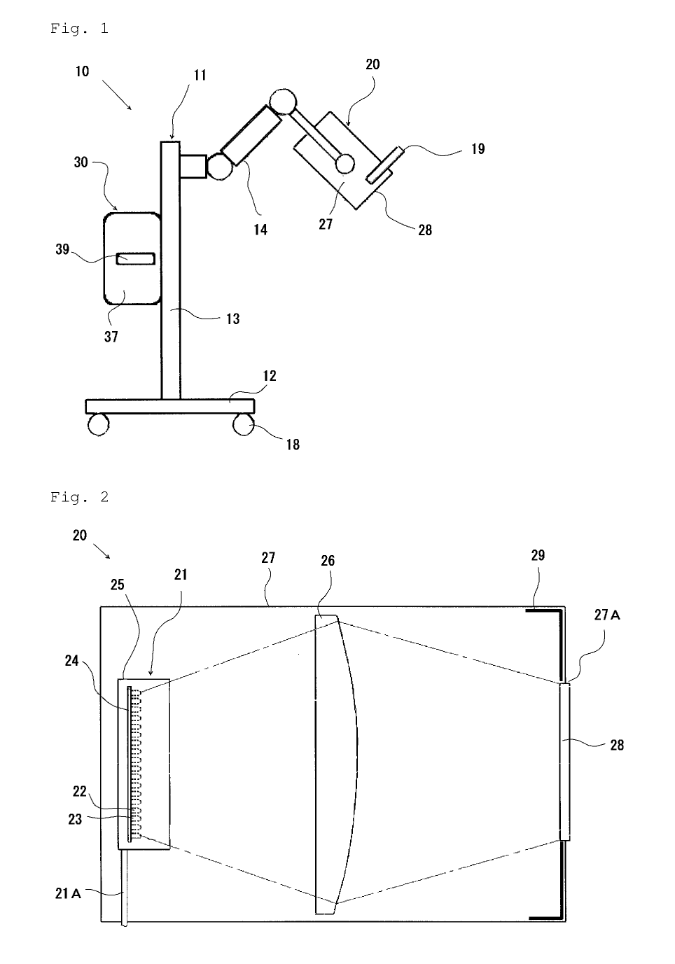

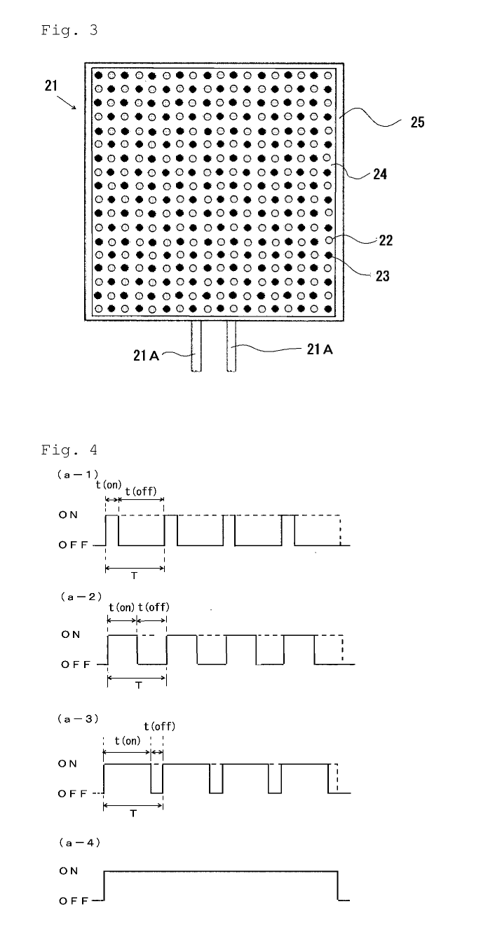

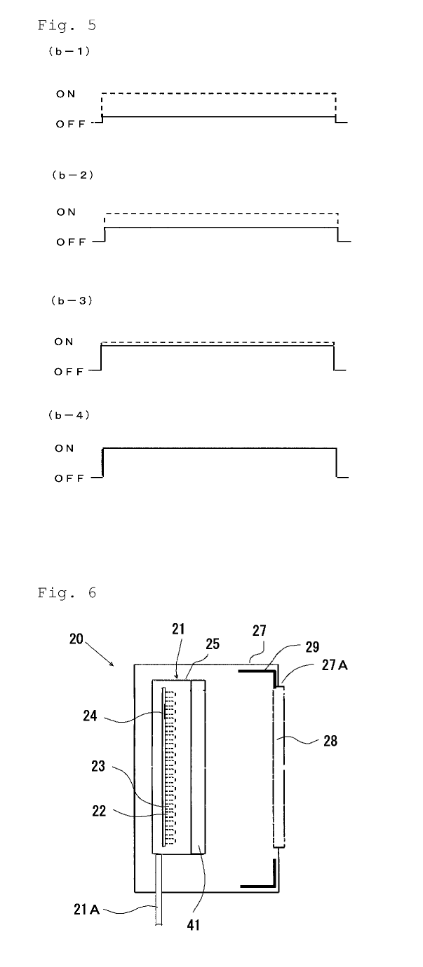

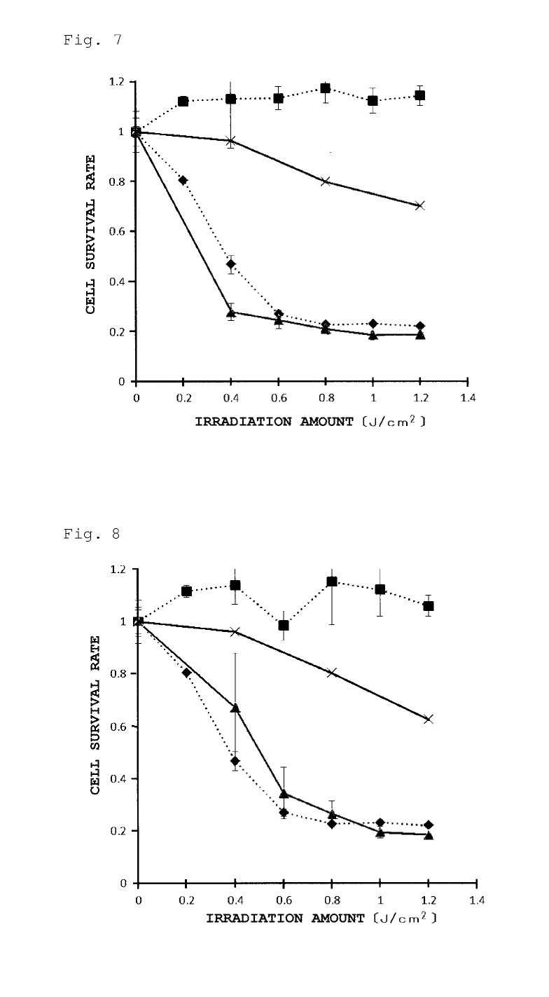

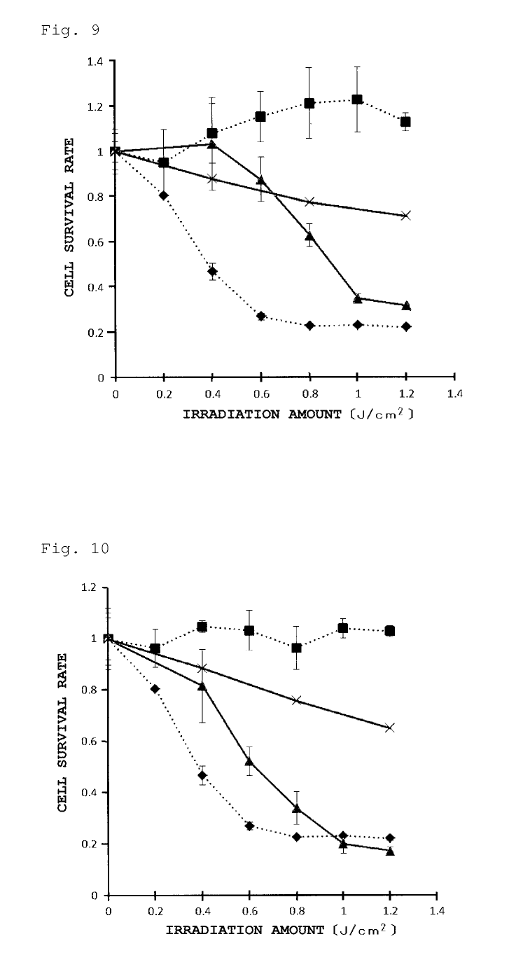

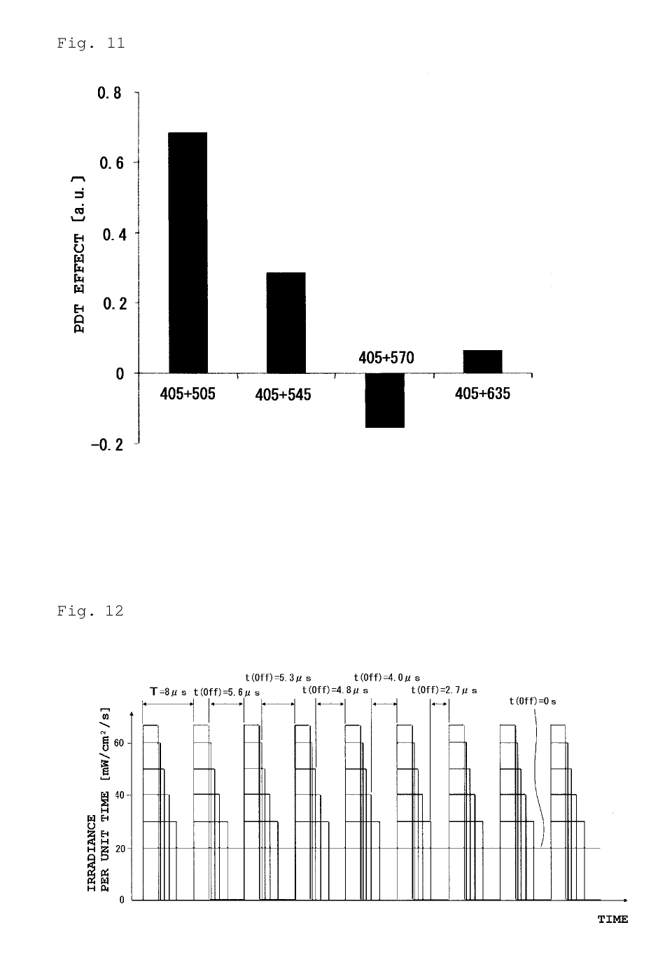

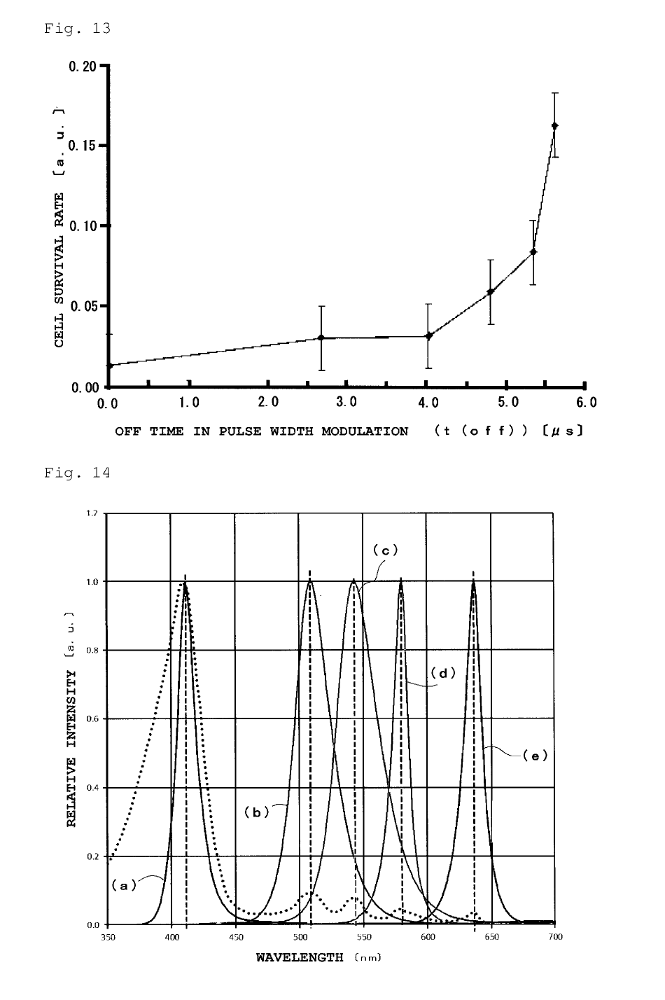

### BRIEF DESCRIPTION OF DRAWINGS

[FIG. 1] is an explanatory view illustrating one example of a configuration of a photodynamic therapy light irradiating device of the present invention.

[FIG. 2] is an explanatory view illustrating a configuration of a light source part in the photodynamic therapy light irradiating device of FIG. 1.

[FIG. 3] is an explanatory view illustrating an arrangement state of LED elements in the light source part of FIG. 2.

[FIG. 4] is an explanatory view illustrating one example of output signals from a control unit.

[FIG. 5] is an explanatory view illustrating another example of output signals from the control unit.

[FIG. 6] is an explanatory view illustrating another example of a configuration of the light source part in the photodynamic therapy light irradiating device of the present invention.

[FIG. 7] is a graph illustrating a relationship between an amount of irradiation and a cell survival rate in single-wavelength irradiation with each of light having a wavelength of 405 nm and light having a wavelength of 505 nm and multiple-wavelength irradiation with light having a wavelength of 405 nm in combination with light having a wavelength of 505 nm, which was obtained in Experimental Example 1.

[FIG. 8] is a graph illustrating a relationship between an amount of irradiation and a cell survival rate in single-wavelength irradiation with each of light having a wavelength of 405 nm and light having a wavelength of 545 nm and multiple-wavelength irradiation with light having a wavelength of 405 nm in combination with light having a wavelength of 545 nm, which was obtained in Experimental Example 1.

[FIG. 9] is a graph illustrating a relationship between an amount of irradiation and a cell survival rate in single-wavelength irradiation with each of light having a wavelength of 405 nm and light having a wavelength of 570 nm and multiple-wavelength irradiation with light having a wavelength of 405 nm in combination with light having a wavelength of 570 nm, which was obtained in Experimental Example 1.

[FIG. 10] is a graph illustrating a relationship between an amount of irradiation and a cell survival rate in single-wavelength irradiation with each of light having a wavelength of 405 nm and light having a wavelength of 635 nm and multiple-wavelength irradiation with light having a wavelength of 405 nm in combination with light having a wavelength of 635 nm, which was obtained in Experimental Example 1.

[FIG. 11] is a graph illustrating a PDT effect in the multiple-wavelength irradiation, which was obtained in Experimental Example 1.

[FIG. 12] is an explanatory view illustrating control using pulse width modulation at 256 modes with a pulse modulation control power supply, which was performed in Experimental Example 2.

[FIG. 13] is a graph illustrating a relationship between an off time in pulse width modulation and a cell survival rate, which was obtained in Experimental Example 2.

[FIG. 14] is a graph illustrating an absorption spectrum of protoporphyrin IX and spectra of the light intensities of five kinds of LED elements each having a peak wavelength corresponding to each of the absorption peaks of the protoporphyrin IX.

### DESCRIPTION OF EMBODIMENTS

Hereinafter, embodiments of the present invention will be described.

FIG. 1 is an explanatory view illustrating one example of a configuration of a photodynamic therapy light irradiating device of the present invention. FIG. 2 is an explanatory view illustrating a configuration of a light source part in the photodynamic therapy light irradiating device of FIG. 1. FIG. 3 is an explanatory view illustrating an arrangement state of LED elements in the light source part of FIG. 2.

A photodynamic therapy light irradiating device 10 is configured to perform a photodynamic therapy (PDT). In the photodynamic therapy, a substance to be administered to the living body including a photosensitizer or a precursor of the photosensitizer is administered to the living body, and the photosensitizer (including the photosensitizer synthesized from a precursor of the photosensitizer in the living body) accumulated in a lesion (lesion abnormal tissue) is irradiated with light.

As the substance to be administered to the living body, a compound that is reacted in the living body if necessary, and is accumulated as a porphyrin compound in the lesion, or the like is adopted.

As examples of the substance to be administered to the living body, may be mentioned δ-aminolevulinic acid (5-ALA). The δ-aminolevulinic acid is the precursor of the photosensitizer as described above. Protoporphyrin IX (PpIX) synthesized through an enzymatic reaction functions as the photosensitizer.

The photodynamic therapy light irradiating device 10 includes a light source part 20 having a first LED element 22 and a second LED element 23, and a control unit 30 of controlling the output of the first LED element 22 and the second LED element 23 that constitute the light source part 20. The light source part 20 and the control unit 30 are supported by a support 11. The support 11 includes a cradle 12 supported through a wheel 18 over a floor surface. At a central portion of the cradle 12, a pillar 13 extends upward. An operation arm 14 supporting the light source part 20 so as to allow the light source part 20 to freely swing with respect to the pillar 13 is provided on the upper portion of the pillar 13. In the support 11, the light source part 20 is attached to the tip of the operation arm 14, and the control unit 30 is attached to the central portion of the pillar 13 by a fixation member (not shown).

In the example illustrated in the drawing, the light source part 20 is provided with a manual lever 19 for manually swinging the light source part 20.

The light source part 20 has two kinds of LED elements, for example, the first LED element 22 having a peak wavelength within a wavelength range of 400 to 420 nm and the second LED element 23 having a peak wavelength within a wavelength range of 500 to 520 nm. When in the light source part 20, the first LED element 22 and the second LED element 23 are operated simultaneously, light from the first LED element 22 (specifically, light having a peak wavelength within a wavelength range of 400 to 420 nm) and light from the second LED element 23 (specifically, light having a peak wavelength within a wavelength range of 500 to 520 nm) are emitted simultaneously.

As shown in FIGS. 2 and 3, it is preferable that the light source part 20 is a light source having a plurality of first LED elements 22 and a plurality of second LED elements 23.

Specifically, the light source part 20 is provided with an LED element unit 21. In the LED element unit 21, the plurality of first LED elements 22 and the plurality of second LED elements 23 are arranged longitudinally and transversely along the outer periphery of a rectangular substrate 24 on the substrate 24 inside a rectangular cylindrical frame 25 as shown in FIG. 3.

The LED element unit 21 is supported by a support member (not shown) inside a rectangular parallelepiped-shaped housing 27 for a light source part having an opening 27A on one side, and disposed so as to face the opening 27A. The LED element unit 21 is electrically connected to a cable 21A for supplying electric power to the first LED elements 22 and the second LED elements 23 that constitute the LED element unit 21. The light source part 20 (the LED element unit 21) is electrically connected to the control unit 30 through the cable 21A. In the housing 27 for a light source part, a lens 26 for collecting and mixing light from the LED element unit 21 (specifically, light from the first LED elements 22 and light from the second LED elements 23) is disposed between the LED element unit 21 and the opening 27A. At a position close to the opening 27A between the lens 26 and the opening 27A, an aperture 29 having a predetermined size is provided. In the housing 27 for a light source part, a window member 28 is provided so as to close the opening 27A. The opening 27A, the aperture 29 and the window member 28 constitute a light output part of the light source part 20.

The light source part 20 is configured so that two different kinds of light, for example, light having a peak wavelength within a wavelength range of 400 to 420 nm (light from the first LED elements 22) and light having a peak wavelength within a wavelength range of 500 to 520 nm (light from the second LED elements 23) are collected and mixed by the lens 26 and emitted from the light output part. Therefore, in the photodynamic therapy light irradiating device 10, one irradiated site is irradiated simultaneously with the light from the first LED elements 22 and the light from the second LED elements 23 that are emitted from the light source part 20.

In the example illustrated in this drawing, an irradiation region on an irradiation surface (irradiated site) has a quadrilateral shape, and has a roughly-estimated size of 100 mm both in longitudinal and transversal directions.

In FIG. 3, the first LED elements 22 are illustrated by coloring with light gray, and the second LED elements 23 are illustrated by coloring with dark gray.

In the LED element unit 21, the number of each of the first LED elements 22 and the second LED elements 23 that constitute the LED element unit 21 is about 100.

In the LED element unit 21, it is preferable that the number of the second LED elements 23 is not lower than the number of the first LED elements 22.

When the number of the second LED elements 23 is not lower than the number of the first LED elements 22, the energy of the light from the first LED elements 22 (light having a peak wavelength within a wavelength range of 400 to 420 nm) and the energy of the light from the second LED elements 23 (light having a peak wavelength within a wavelength range of 500 to 520 nm), of the light from the light source part 20, can be easily adjusted within intended ranges satisfying a relationship between the energies of the light from the first and second LED elements.

In the example illustrated in this drawing, the number of the first LED elements 22 and the number of the second LED elements 23 are the same value as 162.

In the LED element unit 21, it is preferable that the plurality of first LED elements 22 and the plurality of second LED elements 23 are arranged alternately in a lattice shape at a predetermined pitch (distance between centers) so that the same kind of LED elements are not adjacent to each other, as shown in FIG. 3.

When the first LED elements 22 and the second LED elements 23 are arranged alternately in a lattice shape, the illuminance distribution on the irradiation surface is highly uniform. Therefore, even when an actual position of the irradiation surface is shifted forward or backward from a designed center (designed position of the irradiation surface) on an optical axis of the light source part 20, a decrease in mixing degree of the light from the first LED elements 22 with the light from the second LED elements 23 on the irradiation surface (the actual irradiation surface) due to the shifting of position is not caused.

In the example illustrated in this drawing, 162 first LED elements 22 and 162 second LED elements 23 are arranged alternately in a lattice shape (18 columns and 18 rows) at equal intervals on the substrate 24.

For the first LED elements 22, a blue LED element or the like can be adopted.

In the example illustrated in this drawing, a blue LED element having a peak wavelength at 405 nm is adopted for the first LED elements 22. In the blue LED element, a hemispheroidal lens layer made of a transparent resin is provided so as to cover the surface thereof.

For the second LED elements 23, a green LED element or the like can be adopted.

In the example illustrated in this drawing, a green LED element having a peak wavelength at 505 nm is adopted for the second LED elements 23. In the green LED element, a hemispheroidal lens layer made of a transparent resin is provided so as to cover the surface thereof.

As the lens 26, a convex lens, a fresnel lens, or the like can be adopted.

When a fresnel lens is adopted as the lens 26, the size of the light source part 20 can be decreased as compared with a case where a convex lens is adopted as the lens 26. Therefore, a decrease in size of the photodynamic therapy light irradiating device 10 can be achieved.

In the example illustrated in this drawing, a convex lens is adopted as the lens 26.

As the window member 28, a window member having light permeability to light emitted from the LED element unit 21 (specifically, light from the first LED elements 22 and light from the second LED elements 23) and high mechanical strength is adopted.

As examples of a material for the window member 28, may be mentioned quartz glass.

The aperture 29 has a size not more than the opening 27A.

When the aperture 29 is provided to the light source part 20, a boundary between the irradiation region on the irradiation surface and a non-irradiation region can be made clear. Therefore, light irradiation of an unintended part, that is, a part other than the irradiated site (exposure to low-power light) can be prevented.

The control unit 30 is configured to control the outputs of the LED elements (specifically, the first LED elements 22 and the second LED elements 23) constituting the light source part 20.

When the control unit 30 controls the outputs of the LED elements constituting the light source part 20, intended light according to a disease portion or the like can be emitted from the light source part 20.

As specifically described, in the treatment of face, and especially a neoplastic lesion around the eye (specifically, actinic keratosis), irradiation with high-illuminance light is performed. For this reason, there are problems in which an afterimage of light is left in the field of view even under appropriate shading and sufficient treatment satisfaction may not be obtained. Therefore, when the control unit 30 decreases the outputs of the LED elements constituting the light source part 20, such problems can be solved.

It is preferable that the control unit 30 is a control unit capable of separately controlling the output of the first LED elements 22 and the output of the second LED elements 23.

When the control unit 30 is capable of separately controlling the output of the first LED elements 22 and the output of the second LED elements 23, intended light according to the kinds of diseases can be emitted from the light source part 20. Therefore, the light irradiating method of the present invention (specifically, the first light irradiating method of the present invention and the second light irradiating method of the present invention) can be performed.

The light irradiating method of the present invention is a light irradiating method for photodynamic therapy in which one irradiated site is irradiated with light having a peak wavelength within a wavelength range of 400 to 420 nm from the first LED element 22 and light having a peak wavelength within a wavelength range of 500 to 520 nm from the second LED element 23. In the method, the output of any one of the first LED element 22 and the second LED element 23 is made higher than the output of the other. Specifically, in the first light irradiating method of the present invention, the output of light having a peak wavelength within a wavelength range of 400 to 420 nm from the first LED elements 22 is higher than the output of light having a peak wavelength within a wavelength range of 500 to 520 nm from the second LED elements 23. In the second light irradiating method of the present invention, the output of light having a peak wavelength within a wavelength range of 500 to 520 nm from the second LED elements 23 is higher than the output of light having a peak wavelength within a wavelength range of 400 to 420 nm from the first LED elements 22.

In the photodynamic therapy, a light irradiating method to be adopted is appropriately selected according to the kinds of diseases from the first light irradiating method of the present invention and the second light irradiating method of the present invention.

As specifically described, when a lesion is present on a skin surface layer like acne vulgaris, the photodynamic therapy is performed through the first light irradiating method of the present invention. Therefore, the output of the first LED elements 22 is made higher than the output of the second LED elements 23 by the control unit 30. When the output of the light having the peak wavelength within the wavelength range of 400 to 420 nm from the first LED elements 22 is controlled so as to be higher as described above, a higher therapeutic effect can be obtained. This is because the body penetration of the light having the peak wavelength within the wavelength range of 400 to 420 nm is lower than that of the light having the peak wavelength within the wavelength range of 500 to 520 nm.

When a lesion is present at a comparatively deep position in the living body like actinic keratosis and Bowen disease, the photodynamic therapy is performed through the second light irradiating method of the present invention. Specifically, the output of the second LED elements 23 is made higher than that of the first LED elements 22 by the control unit 30. When the output of the light having the peak wavelength within the wavelength range of 500 to 520 nm is controlled so as to be higher as described above, a higher therapeutic effect can be obtained. This is because the body penetration of the light having the peak wavelength within the wavelength range of 500 to 520 nm is higher than that of the light having the peak wavelength within the wavelength range of 400 to 420 nm.

It is preferable that the energy of the light having the peak wavelength within the wavelength range of 500 to 520 nm (the light from the second LED elements 23) is made not lower than the energy of the light having the peak wavelength within the wavelength range of 400 to 420 nm (the light from the first LED elements 22) in light from the light source part 20 by the control unit 30.

In the light from the light source part 20, the energy of the light from the first LED elements 22 and the energy of the light from the second LED elements 23 are appropriately determined according to the kinds of diseases, the state of a lesion, the treatment time (irradiation time), and the like. Specifically, the energy is preferably not lower than 10 mW/cm², more preferably 10 to 60 mW/cm².

It is preferable that the control unit 30 is provided with an irradiation energy adjusting mechanism which adjusts the energy of the light from the light source part 20, for example, the light from the first LED elements 22 (the light having the peak wavelength within the wavelength range of 400 to 420 nm) and the light from the second LED elements 23 (the light having the peak wavelength within the wavelength range of 500 to 520 nm) using pulse width modulation control or amplitude variable control. Therefore, it is preferable that the control unit 30 is provided with an irradiation energy adjusting mechanism using pulse width modulation control or an irradiation energy adjusting mechanism using amplitude variable control as means for controlling the outputs of the LED elements constituting the light source part 20. This irradiation energy adjusting mechanism is configured to be capable of separately adjusting the energy of light from each LED element to separately control the output of the first LED elements 22 and the output of the second LED elements 23. For example, the energy of the light from each LED element can be separately adjusted so that the output of one of the LED elements is 100% and the output of the other is 70%.

In the irradiation energy adjusting mechanism using pulse width modulation control, pulsed-lighting of the LED elements constituting the light source part 20 is performed at high speed to control the duty ratio of pulse wave, as shown in FIGS. 4(a-1) to 4(a-3), and pulsed-lighting of the LED elements constituting the light source part 20 is not performed as shown in FIG. 4(a-4). With this manner, the energy of the light from the light source part 20 is adjusted.

In the irradiation energy adjusting mechanism using amplitude variable control, a current to be supplied to the LED elements constituting the light source part 20 is changed as shown in FIGS. 5(b-1) to 5(b-3) and the current to be supplied to the LED elements constituting the light source part 20 is not changed as shown in FIG. 5(b-4). Thus, the energy of the light from the light source part 20 is adjusted.

(a-1) to (a-4) and (b-1) to (b-4) in FIGS. 4 and 5 are explanatory views illustrating the output signals (drive signals of the LED elements) from the control unit 30. In (a-1) to (a-3) of FIG. 4, an output signal for performing pulse modulation control is represented by a solid line, and an output signal in a case where the pulse modulation control is not performed is represented by a dashed line. Specifically, the solid line in FIG. 4(a-1) represents an output signal for performing the pulse width modulation control at a duty ratio of 10%. The solid line in FIG. 4(a-2) represents an output signal for performing the pulse width modulation control at a duty ratio of 50%. The solid line in FIG. 4(a-3) represents an output signal for performing the pulse width modulation control at a duty ratio of 90%. In (a-1) to (a-3) of FIG. 4, T represents the period of pulse wave, t(on) represents the on time in the pulse width modulation, and t(off) represents the off time in the pulse width modulation. In (b-1) to (b-4) of FIG. 5, an output signal for performing amplitude variable control is represented by a solid line, and an output signal in a case where the control is not performed is represented by a dashed line. Specifically, the solid line in FIG. 5(b-1) represents an output signal for performing the amplitude variable control at an amplitude of 10%. The solid line in FIG. 5(b-2) represents an output signal for performing the amplitude variable control at an amplitude of 50%. The solid line in FIG. 5(b-3) represents an output signal for performing the amplitude variable control at an amplitude of 90%.

(a-4) in FIG. 4 and (b-4) in FIG. 5 each represent the output signal in a case where the control is not performed.

The irradiation energy adjusting mechanism using amplitude variable control is configured, for example, by an amplitude control power supply or the like.

The irradiation energy adjusting mechanism using pulse width modulation control is configured, for example, by a pulse modulation control power supply or the like.

It is preferable that the off time (t(off)) in pulse width modulation is not more than 4 μs when the energy of the light from the light source part 20 (specifically, the light from the first LED elements 22 and the light from the second LED elements 23) are adjusted by the irradiation energy adjusting mechanism using pulse width modulation control. When the light from the first LED elements 22 and the light from the second LED elements 23 are controlled using pulse width modulation by the irradiation energy adjusting mechanism, the off times in pulse width modulation according to each light may be different as long as they are not more than 4 μs.

When the off time in pulse width modulation is not more than 4 μs, a harmful influence such as a decrease in therapeutic effect caused by influence of pulse irradiation, for example, generation of quenching action, does not occur simultaneously, and an excellent therapeutic effect that is the same as that during continuous irradiation under amplitude variable control is obtained.

Herein, the irradiation energy adjusting mechanism using pulse width modulation control may be configured by a pulse width modulation control power supply having a frequency of 125 kHz. In this case, when the pulse width modulation control is performed so that the duty ratio is not more than 50%, the off time (t(off)) in pulse width modulation can be made not more than 4 μs.

In the control unit 30, a power supply unit for driving LEDs, a control unit such as PLC, and the irradiation energy adjusting mechanism (specifically, a pulse modulation control power supply, for example) are disposed inside a rectangular parallelepiped housing 37 for a control unit, and a graphic operation panel 39 is disposed on a side surface of the housing 37 for a control unit.

In the photodynamic therapy light irradiating device 10 with such a configuration, the light source part 20 is disposed away from the irradiated site so that the window member 28 faces the irradiated site. Herein, the distance between the irradiated site and the light source part 20 (window member 28) is preferably 10 to 50 mm from the viewpoint of hygiene and prevention of blurriness of end of irradiated image. For example, the distance may 20 mm. When electric power is supplied to each of the plurality of first LED elements 22 and the plurality of second LED elements 23 from the control unit 30, the LED elements are simultaneously turned on. One irradiated site is irradiated simultaneously with two different kinds of light, specifically, the light from the first LED elements 22 (the light having the peak wavelength within the wavelength range of 400 to 420 nm) and the light from the second LED elements 23 (the light having the peak wavelength within the wavelength range of 500 to 520 nm) in a mixed state.

According to the photodynamic therapy light irradiating device 10, one irradiated site is irradiated with the light having the peak wavelength within the wavelength range of 400 to 420 nm in combination with the light having the peak wavelength within the wavelength range of 500 to 520 nm. Therefore, a synergistic effect due to combination of the two different kinds of light is obtained, as clear from experimental examples described below (for example, Experimental Example 1). The efficiency of treatment by a PDT is exerted. Accordingly, the amount of irradiation (integrated light amount) necessary for the treatment can be decreased as compared with irradiation using a single wavelength with each of the two different kinds of light (specifically, the light having the peak wavelength within the wavelength range of 400 to 420 nm and the light having the peak wavelength within the wavelength range of 500 to 520 nm). As a result, the irradiation time necessary for the treatment can be shortened.

According to the photodynamic therapy light irradiating device 10, an excellent therapeutic effect can thus be obtained by short-time light irradiation.

The reason why the synergistic effect is obtained by combination of the light having the peak wavelength within the wavelength range of 400 to 420 nm and the light having the peak wavelength within the wavelength range of 500 to 520 nm is estimated as follows.

By one of the light having the peak wavelength within the wavelength range of 400 to 420 nm and the light having the peak wavelength within the wavelength range of 500 to 520 nm, a photosensitizer is optically modified. As a result, the photosensitizer has a large absorption peak to the other of the two different kinds of light. The photosensitizer (photomodifiable substance) optically modified by one kind of light is further optically modified by the other kind of light. Thus, it is estimated that the synergistic effect is obtained by combination of the two different kinds of light.

In the photodynamic therapy light irradiating device 10 in which the control unit 30 has the irradiation energy adjusting mechanism using pulse width modulation control, when the off time in pulse width modulation is not more than 4 μs, an excellent therapeutic effect that is the same as that during continuous irradiation under amplitude variable control can be obtained as clear from the below-described experimental example (specifically, Experimental Example 2). Therefore, when in the photodynamic therapy light irradiating device 10, pulse width modulation control that is widely used in industrial applications from the viewpoint of low cost and good distributability is applied, simultaneous occurrence of a harmful influence such as a decrease in therapeutic effect caused by an influence of pulse irradiation (quenching action) can be prevented by setting the off time in pulse width modulation to be not more than 4 μs.

In the photodynamic therapy light irradiating device 10, the control unit 30 is configured such that the output of the first LED elements 22 and the output of the second LED elements 23 are separately controlled. In this case, intended light according to the kinds of diseases or the like can be emitted. Therefore, the light irradiating method of the present invention (specifically, the first light irradiating method of the present invention and the second light irradiating method of the present invention) can be performed.

In the first light irradiating method of the present invention, the output of the light having the peak wavelength within the wavelength range of 400 to 420 nm from the first LED elements 22 is increased. The body penetration of the light having the peak wavelength within the wavelength range of 400 to 420 nm is lower than that of the light having the peak wavelength within the wavelength range of 500 to 520 nm. Therefore, a higher therapeutic effect can be obtained for a disease in which a lesion is present at a comparatively shallow position in the living body by short-time light irradiation.

In the second light irradiating method of the present invention, the output of the light having the peak wavelength within the wavelength range of 500 to 520 nm from the second LED elements 23 is increased. The body penetration of the light having the peak wavelength within the wavelength range of 500 to 520 nm is higher than that of the light having the peak wavelength within the wavelength range of 400 to 420 nm. Therefore, a higher therapeutic effect can be obtained for a disease in which a lesion is present at a comparatively deep position in the living body by short-time light irradiation.

The present invention is not limited to the embodiments described above, and various modifications may be added.

For example, the photodynamic therapy light irradiating device may be a photodynamic therapy light irradiating device capable of selectively turning on only one of the first LED element and the second LED element from the viewpoint of device availability as long as it is capable of turning on both the first LED element and the second LED element.

In the light source part, a diffusion plate 41 may be provided as a light mixing member for mixing the light from the first LED element 22 and the light from the second LED element 23, as shown in FIG. 6. In the photodynamic therapy light irradiating device provided with the light source part 20 having such a configuration, the diffusion plate 41 can be disposed close to the LED element unit 21, as shown in FIG. 6. For example, the diffusion plate 41 is disposed so as to close the opening of the frame 25 in FIG. 6. In this case, a long distance between the LED element unit 21 and the opening 27A of the housing 27 for a light source part is not required, unlike a light source part adopting a lens as the light mixing member as shown in FIG. 2. Therefore, the size of the light source part 20 can be decreased, and a decrease in size of the photodynamic therapy light irradiating device itself can be achieved.

In such a photodynamic therapy light irradiating device, the irradiance is lower by about 30% than that in a photodynamic therapy light irradiating device provided with a lens as a light mixing member. Therefore, the photodynamic therapy light irradiating device is suitably used for a treatment of a disease, in which a necessary amount of light irradiation is low, such as acne vulgaris.

Therefore, it is preferable that the light source part is provided with a lens having a condensing function as alight mixing member for mixing the light from the first LED element and the light from the second LED element, as shown in FIG. 2, to obtain high illuminance on an irradiation surface. However, when in the photodynamic therapy light irradiating device used for a treatment of a disease in which the amount of irradiation necessary for a lesion is comparatively low, high-output LED elements are adopted as the first LED element and the second LED element, a diffusion plate can be suitably used as a light mixing member.

In addition to the first LED element and the second LED element, another LED element such as a red LED element having a peak wavelength at 635 nm may be disposed in the LED element unit of the light source part. According to a photodynamic therapy light irradiating device provided with the light source part having such a configuration, only the red LED element can be selectively turned on. Therefore, the photodynamic therapy light irradiating device can be used as a red light irradiating device. Accordingly, the device availability is enhanced.

Both the first light irradiating method and the second light irradiating method are not limited to use of the photodynamic therapy light irradiating device of the present invention, and can be performed using a device other than the photodynamic therapy light irradiating device of the present invention. For example, the first light irradiating method and the second light irradiating method can be performed using a device provided with the first LED element and a device provided with the second LED element.

Hereinafter, Experimental Examples of the present invention will be described.

### EXPERIMENTAL EXAMPLE 1

In a plurality of plates, 1×10⁵ HaCaT cells (human skin keratinocyte cell line) were cultured over 18 hours, and 200 μL of a δ-aminolevulinic acid (5-ALA) solution having a concentration of 1 mM diluted with phosphate buffered saline (PBS) was added. After 4 hours, the plurality of plates except for one plate were each subjected to five kinds of single-wavelength irradiation and four kinds of multiple-wavelength irradiation (specifically, two-wavelength irradiation) at respective amounts of irradiation of 0.2 J/cm², 0.4 J/cm², 0.6 J/cm², 0.8 J/cm², 1.0 J/cm², and 1.2 J/cm². Specifically, in the five kinds of single-wavelength irradiation, irradiation with each of light having a wavelength of 405 nm, light having a wavelength of 505 nm, light having a wavelength of 545 nm, light having a wavelength of 570 nm, and light having a wavelength of 635 nm was performed. The four kinds of multiple-wavelength irradiation with light in which the light having the wavelength of 405 nm and the light having the wavelength of 505 nm were combined, light in which the light having the wavelength of 405 nm and the light having the wavelength of 545 nm were combined, light in which the light having the wavelength of 405 nm and the light having the wavelength of 570 nm were combined, and light in which the light having the wavelength of 405 nm and the light having the wavelength of 635 nm were combined were performed. In the single-wavelength irradiation and the multiple-wavelength irradiation, as a light source of the light having the wavelength of 405 nm, an LED element in which the energy of light from the light source was 11 mW/cm² (irradiation distance: 100 mm) was used. As a light source of the light having the wavelength of 505 nm, an LED element in which the energy of light from the light source was 17 mW/cm² (irradiation distance: 40 mm) was used.

Subsequently, in the plurality of plates that had been irradiated with light and the plate that had not been irradiated with light, cultivation was performed over 18 hours. The cell survival rate was measured by an MTT assay using an XTT cell proliferation assay kit. The results are shown in FIGS. 7 to 10. In FIGS. 7 to 10, the relative values of cell survival rates relative to the cell survival rate in the plate that had not been irradiated with light were shown.

On the basis of the results of cell survival rate at an amount of irradiation of 0.4 J/cm², a therapeutic effect by a photodynamic therapy (hereinafter also referred to as “PDT effect”) was calculated by the following expression (1). The results are shown in FIG. 11.

In FIG. 7, the results according to the single-wavelength irradiation with the light having the wavelength of 405 nm are plotted as a rhombus (♦), the results according to the single-wavelength irradiation with the light having the wavelength of 505 nm are plotted as a square (▪), and the results according to the multiple-wavelength irradiation with light in which the light having the wavelength of 405 nm and the light having the wavelength of 505 nm are combined are plotted as a triangle (▴).

In FIG. 8, the results according to the single-wavelength irradiation with the light having the wavelength of 405 nm are plotted as a rhombus (♦), the results according to the single-wavelength irradiation with the light having the wavelength of 545 nm are plotted as a square (▪), and the results according to the multiple-wavelength irradiation with light in which the light having the wavelength of 405 nm and the light having the wavelength of 545 nm are combined are plotted as a triangle (▴).

In FIG. 9, the results according to the single-wavelength irradiation with the light having the wavelength of 405 nm are plotted as a rhombus (♦), the results according to the single-wavelength irradiation with the light having the wavelength of 570 nm are plotted as a square (▪), and the results according to the multiple-wavelength irradiation with the light in which the light having the wavelength of 405 nm and the light having the wavelength of 570 nm are combined are plotted as a triangle (▴).

In FIG. 10, the results according to the single-wavelength irradiation with the light having the wavelength of 405 nm are plotted as a rhombus (♦), the results according to the single-wavelength irradiation with the light having the wavelength of 635 nm are plotted as a square (▪), and the results according to the multiple-wavelength irradiation with light in which the light having the wavelength of 405 nm and the light having the wavelength of 635 nm are combined are plotted as a triangle (▴).

In FIGS. 7 to 10, cell survival rate reference values calculated on the basis of the results according to two kinds of single-wavelength irradiation are plotted as a cross (×), and a reference line based on the plotted crosses is shown. Herein, the cell survival rate reference value is a value calculated by the following expression (2). In the expression (2), when I (J/cm²) is an amount of irradiation in multiple-wavelength irradiation, E1 is a cell survival rate in single-wavelength irradiation with light having one wavelength used in the multiple-wavelength irradiation at an amount of irradiation of I/2 (J/cm²), and E2 is a cell survival rate in single-wavelength irradiation with light having the other wavelength used in the multiple-wavelength irradiation at an amount of irradiation of I/2 (J/cm²).

In FIG. 11, a PDT effect according to the multiple-wavelength irradiation with light in which the light having the wavelength of 405 nm and the light having the wavelength of 505 nm are combined is shown as “405+505,” a PDT effect according to the multiple-wavelength irradiation with light in which the light having the wavelength of 405 nm and the light having the wavelength of 545 nm are combined is shown as “405+545,” a PDT effect according to the multiple-wavelength irradiation with light in which the light having the wavelength of 405 nm and the light having the wavelength of 570 nm are combined is shown as “405+570,” and a PDT effect according to the multiple-wavelength irradiation with light in which the light having the wavelength of 405 nm and the light having the wavelength of 635 nm are combined is shown as “405+635.”

PDT effect=1−(cell survival rate) Expression (1):

cell survival rate reference value=(E1+E2)/2 Expression (2):

As clear from the results of Experimental Example 1, the most excellent PDT effect is obtained when among five kinds of light (specifically, the light having the wavelength of 405 nm, the light having the wavelength of 505 nm, the light having the wavelength of 545 nm, the light having the wavelength of 570 nm, and the light having the wavelength of 635 nm) corresponding to five absorption peaks (specifically, wavelengths of 410 nm, 510 nm, 545 nm, 580 nm, and 630 nm) in protoporphyrin IX (PpIX), the light having the wavelength of 405 nm is adopted.

Further, it becomes clear that a synergistic effect is obtained by combination of the light having a wavelength of 405 nm and the light having a wavelength of 505 nm and an excellent PDT effect is obtained even at a low amount of irradiation of 0.4 J/cm².

In all of combinations of the light having the wavelength of 405 nm, and any of the light having the wavelength of 545 nm, the light having the wavelength of 570 nm, and the light having the wavelength of 635 nm, it becomes clear that a synergistic effect is not obtained at a low amount of irradiation of 0.4 J/cm² and a sufficient PDT effect is not obtained.

As specifically described, in the combination of the light having the wavelength of 405 nm and the light having the wavelength of 545 nm and the combination of the light having the wavelength of 405 nm and the light having the wavelength of 635 nm, it becomes clear that a synergistic effect is obtained at an amount of irradiation of not lower than 0.9 J/cm², but the synergistic effect is not obtained at a low amount of irradiation of 0.4 J/cm². In the combination of the light having the wavelength of 405 nm and the light having the wavelength of 570 nm, it becomes clear that a synergistic effect is not obtained at all.

Therefore, according to the photodynamic therapy light irradiating device of the present invention, it has been confirmed that an excellent therapeutic effect is obtained by short-time light irradiation.

### EXPERIMENTAL EXAMPLE 2

In a plurality of plates, 1×10⁵ HaCaT cells (human skin keratinocyte cell line) were cultured over 18 hours, and 200 μL of a δ-aminolevulinic acid (5-ALA) solution having a concentration of 1 mM diluted with phosphate buffered saline (PBS) was added. After 4 hours, the plurality of plates except for one plate were each irradiated with light having a wavelength of 635 nm under conditions (1) to (6) in which the amount of irradiation was 12 J/cm² according to the following table 1. The irradiation was performed using a pulse width modulation control power supply (PWM power supply) with a frequency of 125 kHz capable of adjusting light using pulse width modulation at 256 modes as shown in FIG. 12. Specifically, in the condition (1), light irradiation was performed using pulse width modulation control in which the off time (t(off)) in pulse width modulation was 5.6 μs. In the condition (2), light irradiation was performed using pulse width modulation control in which the off time (t(off)) in pulse width modulation was 5.3 μs. In the condition (3), light irradiation was performed using pulse width modulation control in which the off time (t(off)) in pulse width modulation was 4.8 μs. In the condition (4), light irradiation was performed using pulse width modulation control in which the off time (t(off)) in pulse width modulation was 4.0 μs. In the condition (5), light irradiation was performed using pulse width modulation control in which the off time (t(off)) in pulse width modulation was 2.7 μs. In all the conditions (1) to (5), the pulse period (T) was 8 μs. In the condition (6), light irradiation in which the off time (t(off)) in pulse width modulation was 0 μs, that is, light irradiation that was continuous irradiation under the same condition as that in amplitude variable control was performed.

Subsequently, in the plurality of plates that had been irradiated with light and the plate that had not been irradiated with light, cultivation was performed over 18 hours. The cell survival rate was measured by an MTT assay using an XTT cell proliferation assay kit. The results are shown in FIG. 13. In FIG. 13, the relative values of cell viabilities based on the cell survival rate in the plate that was not irradiated with light were shown.

TABLE 1

| | IRRADIATION | PWM | OFF TIME | IRRADIANCE | AVERAGE | IRRADIATION | IRRADIATION |
|---|---|---|---|---|---|---|---|
| | DISTANCE | CONTROL | (t(off)) | PER UNIT TIME | IRRADIANCE | TIME | AMOUNT |
| | [mm] | STEP | [μs/T] | [mW/cm²/s] | [mW/cm²] | [min] | [J/cm²] |
| CONDITION (1) | 24 | 76 | 5.6 | 67 | 20 | 10 | 12 |
| CONDITION (2) | 32 | 85 | 5.3 | 60 | 20 | 10 | 12 |
| CONDITION (3) | 44 | 102 | 4.8 | 50 | 20 | 10 | 12 |
| CONDITION (4) | 52 | 127 | 4.0 | 40 | 20 | 10 | 12 |
| CONDITION (5) | 71 | 170 | 2.7 | 30 | 20 | 10 | 12 |
| CONDITION (6) | 93 | 255 | 0 | 20 | 20 | 10 | 12 |

As clear from the results of Experimental Example 2, when pulse width modulation control is performed using a pulse width modulation control power supply with a frequency of 125 kHz at a duty ratio of not more than 50% so that the off time (t(off)) is not more than 4 μs, a PDT effect that is the same as that in continuous irradiation using amplitude variable control is obtained.

Therefore, when in a PDT using the photodynamic therapy light irradiating device of the present invention, pulse irradiation is performed using pulse width modulation control, it has been confirmed that an excellent therapeutic effect that is the same as that in continuous irradiation using amplitude variable control is obtained at an off time in pulse width modulation of not more than 4 μs.

### REFERENCE SIGNS LIST

10 photodynamic therapy light irradiating device

11 support

12 cradle

13 pillar

14 operation arm

18 wheel

19 manual lever

20 light source part

21 LED element unit

21A cable

22 first LED element

23 second LED element

24 substrate

25 frame

26 lens

27 housing for light source part

27A opening

28 window member

29 aperture

30 control unit

37 housing for control unit

39 graphic operation panel

41 diffusion plate

1. A photodynamic therapy light irradiating device comprising:
a light source part having a first LED element having a peak wavelength within a wavelength range of 400 to 420 nm and a second LED element having a peak wavelength within a wavelength range of 500 to 520 nm; and
a control unit for controlling outputs of the first LED element and the second LED element, wherein
by operating both of the first LED element and the second LED element that constitute the light source part together, one irradiated site is irradiated with light from the first LED element and light from the second LED element.

2. The photodynamic therapy light irradiating device according to claim 1, wherein an energy of the light emitted from the second LED element is not lower than an energy of the light emitted from the first LED element.

3. The photodynamic therapy light irradiating device according to claim 1, wherein the control unit has an irradiation energy adjustment mechanism for controlling the light emitted from the first LED element and the light emitted from the second LED element using pulse width modulation and an off time in the pulse width modulation is not more than 4 μs.

4. A light irradiating method for a photodynamic therapy comprising irradiating one irradiated site with light having a peak wavelength within a wavelength range of 400 to 420 nm from a first LED element and light having a peak wavelength within a wavelength range of 500 to 520 nm from a second LED element, wherein
an output of the light having the peak wavelength within the wavelength range of 400 to 420 nm from the first LED element is higher than an output of the light having the peak wavelength within the wavelength range of 500 to 520 nm from the second LED element.

5. A light irradiating method for a photodynamic therapy comprising irradiating one irradiated site with light having a peak wavelength within a wavelength range of 400 to 420 nm from a first LED element and light having a peak wavelength within a wavelength range of 500 to 520 nm from a second LED element, wherein
an output of the light having the peak wavelength within the wavelength range of 500 to 520 nm from the second LED element is higher than an output of the light having the peak wavelength within the wavelength range of 400 to 420 nm from the first LED element.
